(19) United States
(12) Patent Application Publication
(10) Pub. No.: US 2006/0271136 A1
(43) Pub. Date: Nov. 30, 2006

(54) Electrical connector to terminate, insulate and environmentally isolate multiple temporary cardiac pacing wires

(76) Inventor: Michael T. Wojciechowicz, Princeton Junction, NJ (US)

Correspondence Address: SYNNESTVEDT LECHNER & WOODBRIDGE LLP, P O BOX 592, PRINCETON, NJ, 08542-0592, US

(21) Appl. No.: 11/200,746

(22) Filed: Aug. 10, 2005

Related U.S. Application Data
(60) Provisional application No. 60/683,944, filed on May 24, 2005.

(51) Int. Cl. A61N 1/05

(52) U.S. Cl. 607/116

(57) ABSTRACT

A connector device, for a temporary cardiac pacing wire, includes a conductive adaptor and a cover section which completely electrically insulates the adaptor and environmentally isolates it. The conductive adaptor comprises a contact receiving end having an aperture therein and a plug portion for plugging into a suitable medical instrument. One or more conductive adaptors are received in a housing comprising a first, or upper section connected by a hinge to a second or lower section. The adaptor is received in the lower section and the plug portion thereof merges inside of a forward barrel portion. A rib in the bottom section cooperates with a pair of ribs on the top section to pinch the pacing wires and hold them in position. A locking post on the inside of the upper cover exerts sufficient frictional stub sections of the electrodes so that they cannot move after the adaptors have been properly positioned inside the lower housing. A cap over the barrel prevents the interior plugs from accidentally contacting an external ground. When the upper section is locked with regard to the lower section, the interior stubs are effectively shielded from exterior strain, electrical contact or dislodgement due to vibration.

### CROSS REFERENCE TO RELATED APPLICATIONS

This application claims the priority of provisional U.S. application Ser. No. 60/683,944 filed on May 24, 2005 and entitled “An Electrical Connector to Terminate, Insulate and Environmentally Isolate Multiple Temporary Cardiac Pacing Wires in Such a Way to Also Allow Direct Connection to External Pacing or Monitoring Equipment” and U.S. application Ser. No. 11/088,586 filed Mar. 24, 2005 entitled “Electrical Connector to Terminate, Insulate and Environmentally Isolate a Temporary Cardiac Pacing Wire” and international application PCT/US04/036325 filed on Oct. 29, 2004 and also entitled “Electrical Connector to Terminate, Insulate and Environmentally Isolate a Temporary Cardiac Pacing Wire” all by Michael T. Wojciechowicz, the entire contents and substance of which are hereby incorporated in total by reference.

### BACKGROUND OF THE INVENTION

1. Field of the Invention

This invention relates to an electrical connector including a conductive section and an insulative cover for connection of a temporary cardiac pacing wire to external equipment.

2. Description of Related Art

Temporary cardiac pacing wires typically have one end attached to the heart during surgery and the opposite end passes through the chest with a portion exterior to the body. To aid in passing through the chest, a temporary cardiac pacing wire has a needle on the end not attached to the heart. A straight needle, also known as a Keith needle, is typically used to penetrate the chest wall from the interior and pull a portion of the temporary pacing wire outside of the body. In order to connect the body exterior end of the temporary pacing wire to pacing and/or monitoring equipment, it is necessary to create an electrical connection on the exterior exposed portion of the temporary pacing wire. Once this step is complete, the temporary pacing wire creates a direct, electrical connection to the surface of the heart. As such, the body exterior termination of the temporary pacing wire should be fully electrically insulated for safety reasons and environmentally isolated from liquids and other contaminates that may degrade its performance both when it is connected to the external equipment and especially when it is not as it is not uncommon for the temporary pacing wire to be unconnected to the external equipment for long periods of time.

The current practice to create this body exterior temporary pacing wire termination utilizes one of two methods.

The first, and most common method, illustrated in FIGS. 1A and 1B, creates the termination by utilizing the already existing Keith needle 12 used to pass the wire 10 through the chest of the patient. The needle 12 is mechanically and electrically attached to the pacing wire 10 during manufacture. After the needle 12 has been passed through the chest, it itself forms the termination. The needle 12 can then be cut at location 16 with a pair of diagonal snips or pliers 14 leaving a stub 18 as the electrical termination, however, the entire needle 12 could also be used as a less convenient “stub”. Alternatively, as shown in FIG. 1B, the needle 12 could include a weakened zone 22 that can be snapped by the hands of a user 20 to produce the stub 18.

The stub 18 is then electrically insulated and sealed from the environment using a combination of improvised materials such as adhesive tape, the finger of a surgical glove, a syringe cover, a tongue depressor, and the like. When it is time for the pacing wire 10 to be connected to the external equipment, the cumbersome insulating and sealing materials must be removed to expose the stub 18. A variety of connecting cables are then used which accept the stub 18 on one end and connect it to external equipment on the other. Unfortunately, the cables do not provide a 100% electrically insulated connection, nor do they protect the connection from the environment.

U.S. Pat. No. 4,442,840, issued to Alexander Wojciechowicz, Jr. on Apr. 17, 1984, teaches an apparatus which can accept the stub pacing wire termination and both electrically insulate and environmentally isolate the stub. However, in order to connect to the external equipment, a proprietary cable must be used. It does not offer a direct connection to the external equipment. The above apparatus taught in U.S. Pat. No. 4,442,840 also uses small removable components which are easily misplaced and once missing cause the electrical insulating and environmentally isolating properties to be eliminated.

A second method of termination is taught by U.S. Pat. No. 4,693,258. It also uses a needle to pass the temporary pacing wire 10 through the chest and exterior of the body. To form its terminal connection, the pacing wire 10 must have a portion of its insulation removed immediately adjacent to the needle 12. The needle 12 is then passed through a hole in an electrically conductive pin. The needle 12 is then cut off and an insulating sleeve is passed around the pin and bare wire. The compression of the sleeve around the pin maintains the electrical contact between the exposed conductor of the pacing wire and the pin. This method allows direct connection to the external equipment, however, it does not create an electrically insulated or environmentally isolated connection as the portion of the pin which does not plug into the external equipment is uncovered and often times the uninsulated conductor of the pacing wire is also exposed due to errors in the placement of the cut when removing the needle during assembly of the apparatus.

U.S. Pat. No. 6,644,998, entitled “Electrical Connecting Element”, issued to Kaufmann et al describes therein a connector for a wire lead extending from the heart muscle, out of the body, and towards an external heart pacemaker. Apparently there is no needle on the end of the wire lead which passes through the exterior of the patient's body. Accordingly, this connector engages a lead end, rather than a needle, connected to a lead end. The connector is intended to engage an insulated wire lead end which is inserted through a clamping sleeve and into engagement with a squeezing contact element held by an insulation sleeve which mates with the clamping sleeve. The mating of the clamping sleeve and the insulation sleeve forces a squeezing contact element into the insulation making electrical contact with the wire lead.

Similarly, U.S. Pat. No. 6,397,108, entitled “Safety Adaptor for Temporary Medical Leads” and issued to Camps et al describes an adaptor for attaching a temporary cardiac pacing lead or analogous lead to a pacemaker or other apparatus exterior to the patient. According to one embodiment of the invention, an elongate assembly comprises a body and a door hinge together along a side edge that cooperates to receive and contain a lead including a Keith-type needle which is broken off after the lead end is inserted in the assembly. The assembly is provided with contacts which enable the assembly, upon insertion into a receptacle exterior to the apparatus, to communicate signals between the exterior apparatus and the lead contained in the assembly. The Camps, et al assembly includes two (2) moveable sections connected by a hinge pin which allows an adaptor arm to move in and out of position as shown in FIGS. 3, 4, 6 and 7 thereof. When the adaptor arm is in the closed position as shown in FIG. 7, the latch helps to lock and unlock the assembly when inserted into an appropriate electronic device.

U.S. Pat. No. 6,021,355, entitled “Surgical Electrode Having a Partially Insulated Needle”, issued to Slichervinsky, discloses a device in which the insulation protected end of a temporary cardiac pacing wire exits the patient's body and is intended for connection to an external pacemaker.

U.S. Pat. No. 5,350,419, entitled “Cardiac Pacing Lead”, issued to Bendel, at al describes a device which provides a direct connection between the remaining portion of a Keith-type needle and an exterior apparatus.

U.S. Pat. No. 6,254,425, entitled “Electrical Connector for Cardiac Devices”, issued to Slichervinsky et al, likewise describes a temporary cardiac pacing wire, including multiple conductive sections, wherein a Keith-type needle is broken to create a segment to plug into an exterior pacemaker. A branch electrode wire is inserted into a blind hole at one end of an elongate conductive wire segment to form a connector which also plugs into the pacemaker.

U.S. Patent Application Publication US 2003/0040784- A1, published on Feb. 27, 2003, and entitled “Medical Lead Adapter for Storing, Isolating, Identifying and Connecting Temporary Pacing Leads” is of general interest and teaches background that relates to the general state of the prior art.

U.S. Pat. No. 5,795,178 entitled “Plug For Heart Electrode Wire” illustrates, in FIGS. 9, 10A, 10B, 10C and 10D how a peg may be used to hold a wire in place in a heart electrode assembly.

U.S. Pat. No. 5,983,142 entitled “Temporary Pacing Wire for Use in Cardiac Surgery” illustrates how a male lead can be inserted into a female receptor, while simultaneously providing a water-tight seal.

U.S. Pat. No. 6,968,082 entitled “Pacemaker Lead Locking Mechanism” describes another device for locking a pacing wire securely into an electrode assembly.

The following patents are cited as showing general state-of-the-art of less relevance to the present invention: 3,760,332; 4,214,594; 4,630,617; 4,784,141; 4,995,389; 5,378,177; 5,476,497; 5,679,022; 5,782,892; and, 6,501,990.

Lastly, a different approach to a similar problem is described in international application PCT/US04/03625 filed on Oct. 24, 2004 by the present inventor, Michael T. Wojciechowicz, and its national phase counterpart U.S. patent application Ser. No. 11/088,586 filed on Mar. 24, 2005 and still pending.

While the prior art discloses a variety of different devices for connecting a lead to a temporary cardiac pacing device, nevertheless, there does not appear to be many truly efficient and effective connectors to terminate, insulate, and environmentally isolate a temporary cardiac pacing wire to the satisfaction of the surgeons that employ them and the patients who must live with them.

It was in the context of the foregoing prior art that the present invention arose.

### SUMMARY OF THE INVENTION

Briefly described, the invention comprises an electrical connector for terminating, insulating and environmentally isolating a temporary cardiac pacing wire. The pacing wire may either be the end of the wire itself or, perhaps, a broken off Keith-type needle. The invention includes two primary parts, namely, a conductive adaptor body and an insulated housing. The conductive adaptor body has a contact receiving section, including an aperture therein for receiving the wire or stub end of the Keith-needle, and a plug section for connection to a medical device. An insulated housing or cover includes a first and second, upper and lower, section connected together by a hinge at the ends of the upper and lower sections for surrounding the contact receiving section of the conductive adaptor body and, further, includes a cap connected by a tether or a web to the lower housing. The lower housing includes a pair of posts that engage with a pair of apertures in the upper housing to lock the upper and lower housing together during use. A pair of ribs inside the upper housing cooperate with a rib inside the lower housing to pinch the cardiac pacing wires so that they cannot be accidentally pulled out of the housing. In addition, the upper housing includes another internal rib that pushes down on the needle stubs against a support rib located in the lower housing thereby exerting frictional force on the stubs when the housing is closed so that the stubs don't move laterally or longitudinally and maintain electrical contact with the adaptor body when the upper and lower housings are properly locked with respect to each other. When the entire conductive adaptor body is covered and the cap is in place, the conductive adaptor body is completely electrically insulated and environmentally isolated from the exterior so that no stray currents can affect the patient's heart. The cap portion, however, can be selectively removed, without removing the entire cover, so that the plug may be plugged into an electrical apparatus. The adaptor is firmly held in place in the cover so that the plug can be inserted, even upside down, without risk of separating from the cover. When the cap is removed, the device can be plugged into a medical instrument, also without fear of electrical or environmental contamination.

The above invention may be further understood by reference to the following drawings.

### BRIEF DESCRIPTION OF THE DRAWINGS

FIG. 1A illustrates the manner in which a Keith-type needle can be cut by a pair of diagonal pliers so as to produce a stub for insertion into the preferred embodiment of the invention.

FIG. 1B illustrates an alternative procedure for producing a stub in which a Keith-type needle is broken at a weakened zone so as to produce an appropriate stub.

FIG. 2A is a perspective view of the conductive adaptor body.

FIG. 2B is a cross-sectional view of the conductive adaptor body.

FIG. 2C illustrates the manner in which a needle, needle stub, or uninsulated wire is inserted into the aperture in the rear of the contact receiving section of the conductive adaptor body.

FIG. 2D illustrates the manner in which the needle, needle stub or uninsulated wire is finally received in the contact receiving section of the conductive adaptor body after the procedure initiated in FIG. 2C is completed.

FIG. 3A is a general cross-sectional schematic showing the combination of the pacing wire and stub.

FIG. 3B is a front-elevational view of a typical connector located on cardiac instrumentation that mates with the plug that is the subject matter of the preferred embodiment of the invention.

FIG. 4A illustrates the preferred embodiment of the invention with the first and second sections open and the cover portion removed.

FIG. 4B illustrates the preferred embodiment of the present invention with the first and second sections closed and the cover removed.

FIG. 5A is a perspective view of the preferred embodiment of the electrical connector invention with the first and second sections open showing the pacing wires and the stub sections in operating position.

FIG. 5B is a rear-perspective view of the preferred embodiment of the invention shown in FIG. 5A.

FIG. 6A is a front-perspective view of the preferred embodiment of the invention with the first and second sections of the invention shown in the closed and locked position.

FIG. 6B is a rear-perspective view of the preferred embodiment of the invention as seen in FIG. 6A.

FIG. 7 is a left side, cross-sectional view of the preferred embodiment of the invention shown in the closed and locked state as seen in FIGS. 6A and 6B.

FIG. 8 is a top, plan, cross sectional view of the preferred embodiment of the connector seen in FIG. 7.

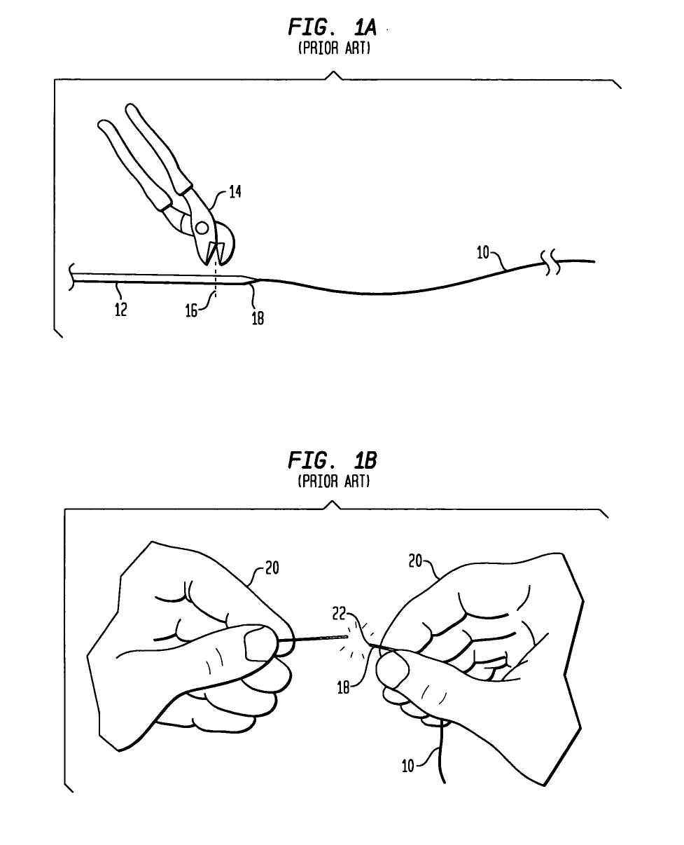

### DETAILED DESCRIPTION OF THE PREFERRED EMBODIMENT

During the course of this description like numbers will be used to identify like elements according to the different views which illustrate the invention.

The present invention comprises an easy-to-use connector which electrically insulates and environmentally isolates the body exterior stub 18 of a temporary cardiac pacing wire 10 and allows it to be directly connected to external equipment without the need for additional cables or interfaces. There is currently no technology which achieves these goals in a satisfactory manner.

The present invention consists of two main components, namely, an electrically conductive structure 30 and a housing 40.

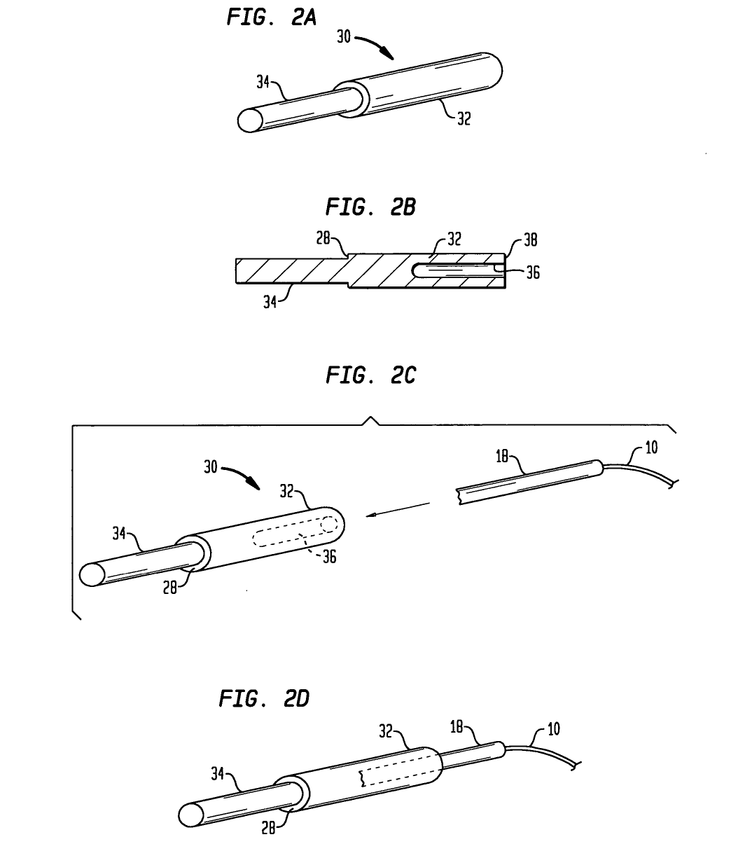

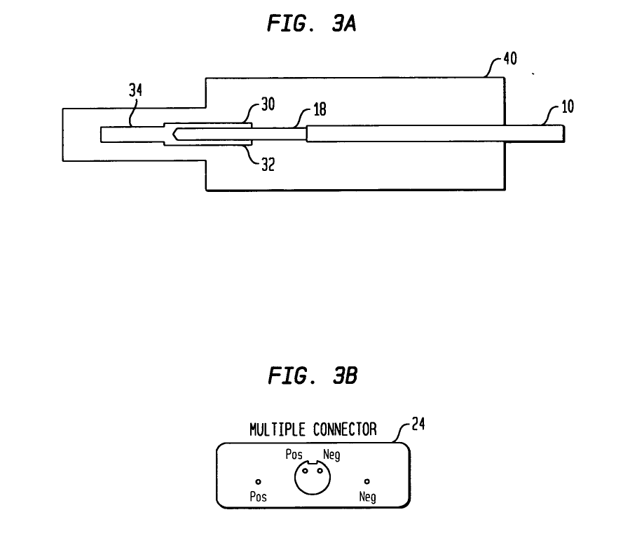

As shown in FIGS. 2A, 2B and 3A the electrically conductive structure of adaptor 30 includes a contact receiving end portion 32 and a plug section 34 separated by a front-facing surface 28. Adaptor 30 also includes a rear-facing surface 38 and an aperture, hole, slot, etc. 36 for accepting the stub 18, or needle 12 or bare connector 10 of the cardiac pacing wire 10. The aperture 36 can also be a groove, channel or other method for accepting a stub 18, needle 12 or wire 10. FIG. 2C illustrates the manner in which a stub 18 is inserted into the aperture 36 of the conductive adaptor 30. FIG. 2D illustrates the conductive adaptor 30 after the stub 18 has been fully received in the aperture 36.

In summary, the conductive adaptor 30 comprises a male pin 34 sized to fit the female receptacle of the external equipment. The opposite end of the structure 30 includes an aperture 36, which could be a passageway, hole, tapered hole, groove, channel, or any other aperture, for accepting the stub 18 or uninsulated wire portion 10 of a temporary cardiac pacing wire in such a way as to create an electrically conductive union. While the preferred embodiment of the conductive adaptor 30 is a single piece structure, it could alternatively be made by the mating of two or more conductive pieces.

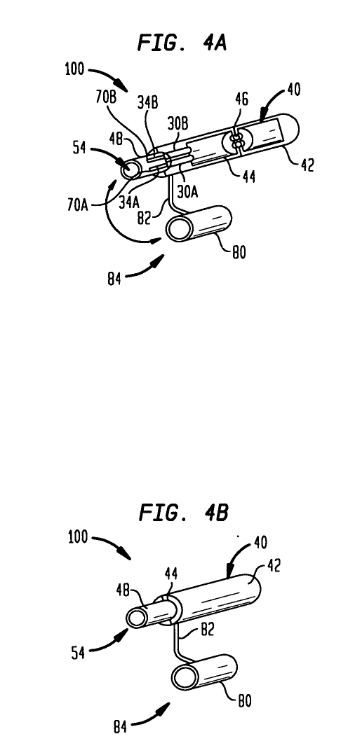

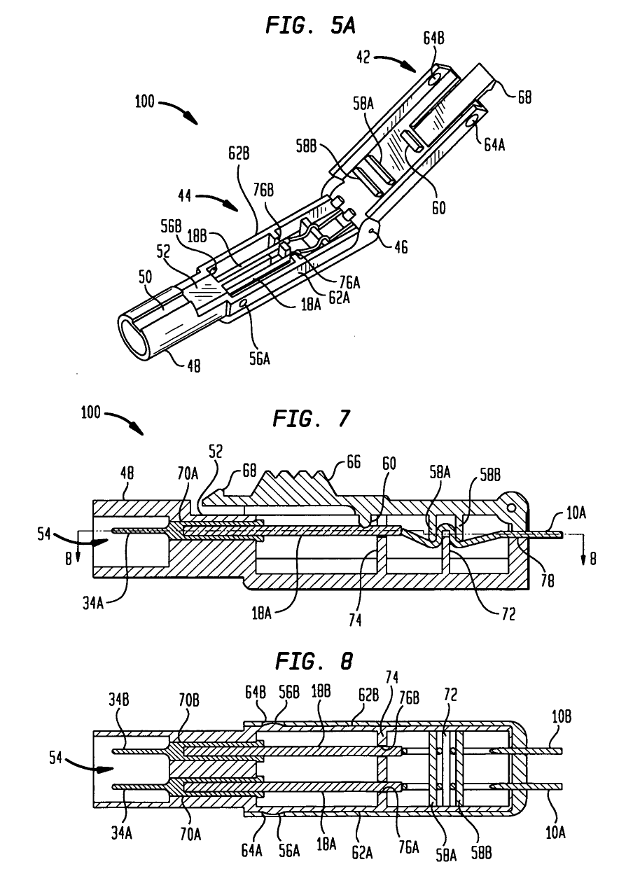

FIG. 3B is a front-elevational view of the female portion of a conventional connector 24 such as is found on typical cardiac instrumentation. This portion of the connector 24 is intended to mate with the preferred embodiment of the invention illustrated in FIGS. 4A-7.

The preferred embodiment of the invention is shown in various detail in FIGS. 4A - 7. The preferred embodiment 100 is illustrated in FIG. 4A in the open position and includes a connector housing 40 comprising a first, or upper housing 42 and a second, or lower housing 44 connected by a hinge pin 46.

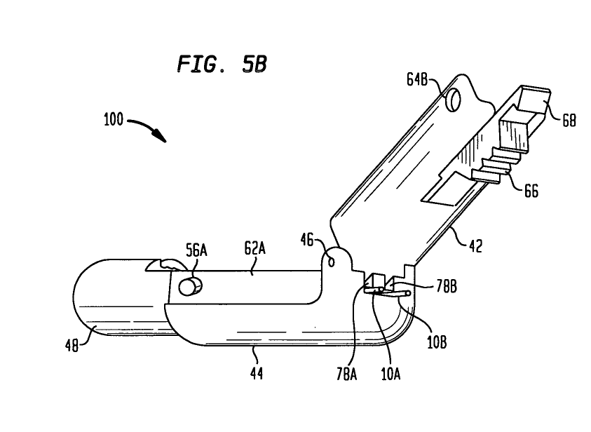

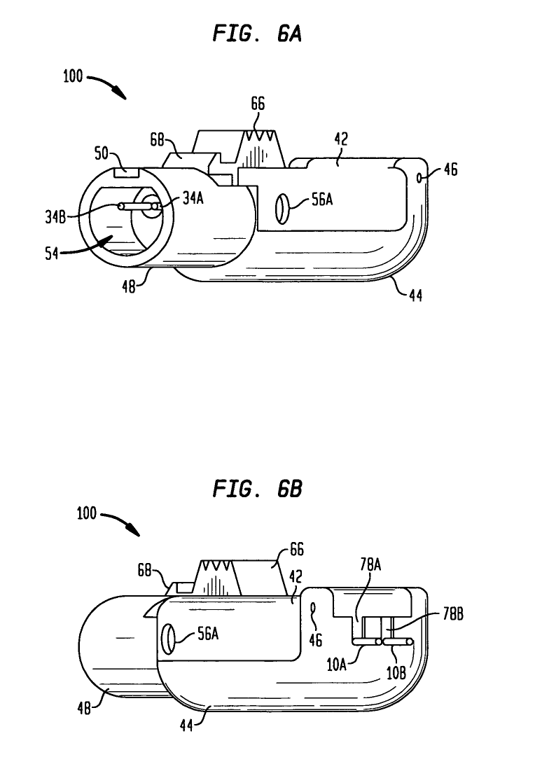

The lower housing 44 includes a barrel section 48 at the forward end thereof. The barrel section includes a cavity therein for protecting electrode pins 34A and 34B. A groove 50 on the top of barrel 48, as shown in FIG. 5A, mates with the complimentary portion of the connector 24 on the electronic equipment as shown in FIG. 3B. Also located on the top of barrel 48 is a flat portion 52, also seen in FIG. 5A, which accommodates the equipment engaging tab 68 on knob 66 on the first, upper housing section 42. A pair of locking posts 56A and 56B located on opposite sidewalls 62A and 62B of the lower housing 44 are intended to engage with and lock in apertures 64A and 64B, respectively in the upper housing 42 when the invention 100 is in the closed mode as best seen in FIGS. 6A, 6B and 7.

The lower section 44 includes an interior cavity having a lower pacing wire capture and strain relief rib 72 and a lower electrode support rib 74 therein as best seen in the cross-section of FIGS. 7 and 8. Lower electrode support rib 74 includes a pair of notches 76A and 76B which support the ends of the stubs 18A and 18B closest to the pacing wire 10A and 10B, respectively, as seen in FIGS. 5B and 6B. The other end of the stubs 18A and 18B is received in contact receiving apertures 70A and 70B in the forward section of the interior cavity of the lower 30 housing 44. In other words, stubs 18A and 18B are supported by apertures 70A and 70B at one end and by notches 76A and 76B in the lower electrode support rib 74 at the other. While this support is good, nevertheless, there is the potential that the stubs 18A and 18B can accidentally slip away from apertures 70A and 70B thereby causing electrical connection difficulties. This difficulty can be substantially reduced, if not eliminated, by employing an electrode locking post 60 on the inside of the upper housing section 42. When the invention 100 is in the closed mode, as shown in FIGS. 7 and 8, the electrode locking post 60 contacts the stubs 18A and 18B and effectively, by force of friction, locks them in position so that they cannot move laterally or longitudinally inside of apertures 70A and 70B and remain in electrical contact.

The first, or upper, section 42 includes, at its forward end an equipment engagement tab 68 and an equipment engagement/disengagement knob 66. Engagement tab 68 mates with the female connector 24 illustrated in FIG. 3B. The underside of the upper section 42 includes a pair of pacing wire capture and strain relief ribs 58A and 58B which cooperate with lower rib 72 to pinch and capture the pacing wires 10A and 10B as shown in FIG. 7. In essence, ribs 58A and 58B pinch the wires 10A and 10B on opposite sides of the lower pacing wire capture and strain relief rib 72. The pacing wires 10A and 10B pass through slots 78A and 78B, as seen, for example in FIGS. 5B and 6B.

It is very important that any strain placed upon the pacing wires 10A and 10B not be transmitted to the stubs 18A and 18B so that they become dislodged from apertures 70A and 70B, respectively, or that they separate from the male pins 34A and 34B thereby causing electrical shorts or disconnects. This problem is avoided in several ways. First, ribs 58A and 58B cooperate with rib 72 to pinch the pacing wires 10A and 10B so that a force placed on the pacing wires 10A and 10B is not transmitted to the stubs 18A and 18B. Second, the locking post 60 on the upper section 42 pushes down on the electrode stubs 18A and 18B against the lower rib 74 and the apertures 70A and 70B so as to effectively lock the stubs 18A and 18B in place so that they cannot accidentally slip out of contact with the adaptors 30A and 30B once they are placed properly inside the lower section 44 and the upper section 42 is closed and locked in position, in which case the locking posts 56A and 56B are securely received and locked in apertures 64A and 64B as seen, for example, in FIGS. 6A, 6B, 7 and 8. Third, the interior cavity 54 inside the barrel 48 protects the male pins 34A and 34B from accidental contact when the cap 80 is removed. Finally, the cap 80, which includes interior cavity 84 is connected by a tether 82 as shown in FIGS. 4A and 4B. When the cap 80 is in position on top of the barrel 48, it absolutely keeps the electrode plugs 34A and 34B from coming into contact with the outside world and thereby causing a short or electrically problem.

While the invention has been described with reference to the preferred embodiment thereof, it will be appreciated by those of ordinary skill in the art that various modifications can be made to the structure and elements of the invention without departing from the spirit of the invention as a whole.

I claim:

1. A connector apparatus for accepting at least one temporary cardiac pacing wire including a conductive adaptor body having a contact receiving section including an aperture therein for receiving a stub from a pacing wire attached to the heart and a plug section for connection to a medical device, said apparatus comprising:
a first housing having at least one aperture therein for receiving said conductive adaptor body;
a second housing for mating with said first housing and for enclosing said stub;
a hinge for connection said first and second housing sections together so that said apparatus can be opened and closed; and,
holding means to prevent said stub from being accidentally dislodged from said apparatus when said apparatus is closed.

2. The apparatus of claim 1 wherein said holding means comprises:
wire holding means for contacting said pacing wire when said apparatus is closed and preventing stress when applied to said pacing wire from being applied to said stub.

3. The apparatus of claim 2 wherein said holding means further comprises:
a stub engaging means for contacting and immobilizing said stub when said apparatus is closed.

4. The apparatus of claim 3 wherein said stub engaging means comprises a rib located on one of said housings that only contacts and engages said stub when said apparatus is in the substantially closed position,
wherein the force applied to said stub by said rib immobilizes said stub so that it can't back out of electrical contact with said adaptor body.

5. The apparatus of claim 4 wherein said wire holding means comprises at least one wire engaging rib on said first housing sections and at least one wire engaging rib on said second housing engaging section,
wherein when said first and second sections are closed said wire engaging ribs cooperate to pinch said wire thereby preventing stress applied to said wire from outside of said housing from being transmitted to said stub.

6. The apparatus of claim 5 further comprising:
a barrel section having a cavity therein and connected to one of said housing sections for surrounding said plug section so that said plug section is received in said cavity and protected from accidental contact.

7. The apparatus of claim 6 further comprising:
a cap for covering barrel section and thereby protecting said plug section of said conductive adaptor body.

8. The apparatus of claim 7 further comprising:
connector means for flexibly attaching said cap to one of said housing sections,
wherein said connector means prevents said cap from becoming unintentionally separated from said apparatus.
